(19) United States
(12) Patent Application Publication
(10) Pub. No.: US 2025/0187910 A1
(43) Pub. Date: Jun. 12, 2025

(54) METHODS OF TRANSPORTING HYDROGEN - FISCHER-TROPSCH REACTIONS AND DEHYDROGENATION AT SEPARATE CHEMICAL PROCESSING FACILITIES

(71) Applicant: Saudi Arabian Oil Company, Dhahran (SA)

(72) Inventors: Guanghui Zhu, Dhahran (SA); Anas S. Al-Aqeeli, Dhahran (SA); Faisal M. Almulla, Dhahran (SA); Raed H. Abudawoud, Dhahran (SA)

(73) Assignee: Saudi Arabian Oil Company, Dhahran (SA)

(21) Appl. No.: 18/535,132

(22) Filed: Dec. 11, 2023

(51) Int. Cl. C01B 3/00

(52) U.S. Cl. CPC C01B 3/0015; C01B 2203/0233; C01B 2203/062; C01B 2203/1235

(57) ABSTRACT

A method of transporting hydrogen may comprise, at a first chemical processing facility, passing carbon monoxide and hydrogen gas to a first reactor, wherein the carbon monoxide and the hydrogen gas undergo a Fischer-Tropsch reaction in the first reactor and form a mixed hydrocarbon product. The method may further comprise transporting at least a portion of the mixed hydrocarbon product from the first chemical processing facility to a second chemical processing facility, and at the second chemical processing facility, dehydrogenating at least a portion of the mixed hydrocarbon product to form a hydrogen gas product. The first chemical processing facility and the second chemical processing facility may be separated by a distance of at least 100 km.

### TECHNICAL FIELD

Embodiments of the present disclosure generally relate to hydrogen processing, and more specifically, to methods and systems utilized to transport hydrogen.

### BACKGROUND

Hydrogen is growing in importance as an environmentally friendly precursor chemical and fuel. Processes for the production and usage of hydrogen are relatively well developed. However, processes for the storage and transportation of hydrogen are still insufficient to meet the needs of the hydrogen industry. Generally, hydrogen is stored and transported in the form of compressed gaseous hydrogen molecules (e.g., at above 5,000 pounds per square inch). However, these conventional gaseous hydrogen transportation techniques are costly and inefficient. For example, the compression process consumes a large amount of energy (estimated to be 30% or more of the energy content of the hydrogen). Also, transport and storage of the compressed hydrogen requires expensive pressure vessels. Some of the hydrogen molecules can even escape through the walls of hydrogen containment vessels. The hydrogen can also cause embrittlement of the storage and transport vessels. Overall, better methods of hydrogen storage and transport are needed.

### BRIEF SUMMARY

Embodiments of the present disclosure provide methods of transporting hydrogen utilizing Fischer-Tropsch synthesis. In the methods provided, at a first chemical processing facility, carbon monoxide and hydrogen gas undergo a Fischer-Tropsch reaction to form a mixed hydrocarbon product that is more easily transportable than hydrogen gas. The hydrogen atoms from the hydrogen gas are thus embodied in the more easily transportable mixed hydrocarbon product. The mixed hydrocarbon product, or a portion thereof, is then transported to a second chemical processing facility where it is dehydrogenated to form a hydrogen gas product. The hydrogen atoms initially in the hydrogen gas are, thus, transported between chemical processing facilities. In such embodiments, the hydrogen atoms may be transported over vast distances, such as between countries or continents, without the need for costly hydrogen gas pressurization.

According to one or more embodiments, a method of transporting hydrogen may comprise, at a first chemical processing facility, passing carbon monoxide and hydrogen gas to a first reactor, wherein the carbon monoxide and the hydrogen gas undergo a Fischer-Tropsch reaction in the first reactor and form a mixed hydrocarbon product. The method may further comprise transporting at least a portion of the mixed hydrocarbon product from the first chemical processing facility to a second chemical processing facility, and at the second chemical processing facility, dehydrogenating at least a portion of the mixed hydrocarbon product to form a hydrogen gas product. The first chemical processing facility and the second chemical processing facility may be separated by a distance of at least 100 km.

These and other embodiments are described in more detail in the Detailed Description. It is to be understood that both the foregoing general description and the following detailed description present embodiments of the presently disclosed technology, and are intended to provide an overview or framework for understanding the nature and character of the technology as it is claimed. The accompanying drawings are included to provide a further understanding of the presently disclosed technology and are incorporated into and constitute a part of this specification. The drawings illustrate various embodiments and, together with the description, serve to explain the principles and operations of the presently disclosed technology. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 schematically depicts a diagram of a system for transporting hydrogen, according to one or more embodiments described in this disclosure;

FIG. 2 schematically depicts a diagram of another system for transporting hydrogen, according to one or more embodiments described in this disclosure;

FIG. 3 schematically depicts a diagram of yet another system for transporting hydrogen, according to one or more embodiments described in this disclosure;

FIG. 4 schematically depicts a diagram of yet another system for transporting hydrogen, according to one or more embodiments described in this disclosure; and

FIG. 5 schematically depicts a diagram of yet another system for transporting hydrogen, according to one or more embodiments described in this disclosure.

For the purpose of describing the simplified schematic illustrations and descriptions of the relevant figures, the numerous valves, temperature sensors, electronic controllers and the like that may be employed and well known to those of ordinary skill in the art of certain chemical processing operations are not included. Further, accompanying components that are often included in typical chemical processing operations, such as air supplies, catalyst hoppers, and flue gas handling systems, are not depicted. However, operational components, such as those described in the present disclosure, may be added to the embodiments described in this disclosure.

It should further be noted that arrows in the drawings refer to process streams. However, the arrows may equivalently refer to transfer lines which may serve to transfer process streams between two or more system components. Additionally, arrows that connect to system components define inlets or outlets in each given system component. The arrow direction corresponds generally with the major direction of movement of the materials of the stream contained within the physical transfer line signified by the arrow. Furthermore, arrows which do not connect two or more system components signify a product stream which exits the depicted system or a system inlet stream which enters the depicted system. Product streams may be further processed in accompanying chemical processing systems or may be commercialized as end products. System inlet streams may be streams transferred from accompanying chemical processing systems or may be non-processed feedstock streams. Some arrows may represent recycle streams, which are effluent streams of system components that are recycled back into the system. However, it should be understood that any represented recycle stream, in some embodiments, may be replaced by a system inlet stream of the same material, and that a portion of a recycle stream may exit the system as a system product.

Additionally, arrows in the drawings may schematically depict process steps of transporting a stream from one system component to another system component. For example, an arrow from one system component pointing to another system component may represent “passing” a system component effluent to another system component, which may include the contents of a process stream “exiting” or being “removed” from one system component and “introducing” the contents of that product stream to another system component. It should be understood that arrows in the relevant figures are not indicative of necessary or essential steps.

It should be understood that according to the embodiments presented in the relevant figures, an arrow between two system components may signify that the stream is not processed between the two system components. In other embodiments, the stream signified by the arrow may have substantially the same composition throughout its transport between the two system components. Additionally, it should be understood that in one or more embodiments, an arrow may represent that at least 75 wt. %, at least 90 wt. %, at least 95 wt. %, at least 99 wt. %, at least 99.9 wt. %, or even 100 wt. % of the stream is transported between the system components. As such, in some embodiments, less than all of the streams signified by an arrow may be transported between the system components, such as if a slip stream is present.

It should be understood that two or more process streams are “mixed” or “combined” when two or more lines intersect in the schematic flow diagrams of the relevant figures. Mixing or combining may also include mixing by directly introducing both streams into a like reactor, separation device, or other system component. For example, it should be understood that when two streams are depicted as being combined directly prior to entering a separation unit or reactor, that in some embodiments the streams could equivalently be introduced into the separation unit or reactor and be mixed in the reactor.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

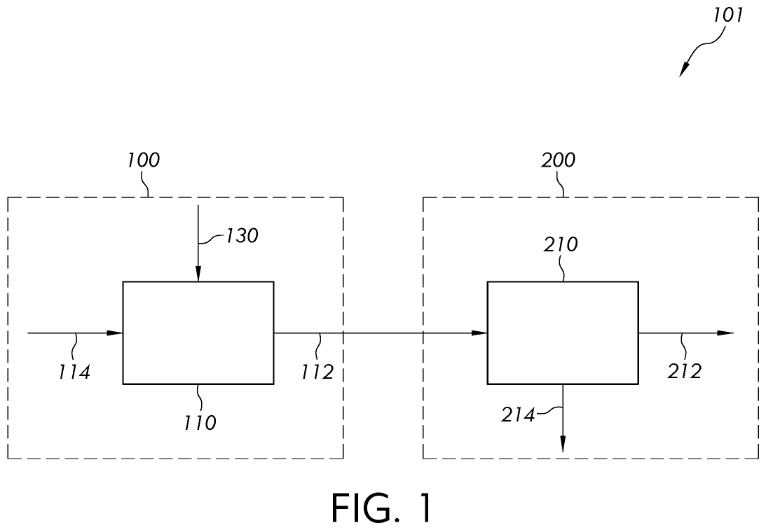

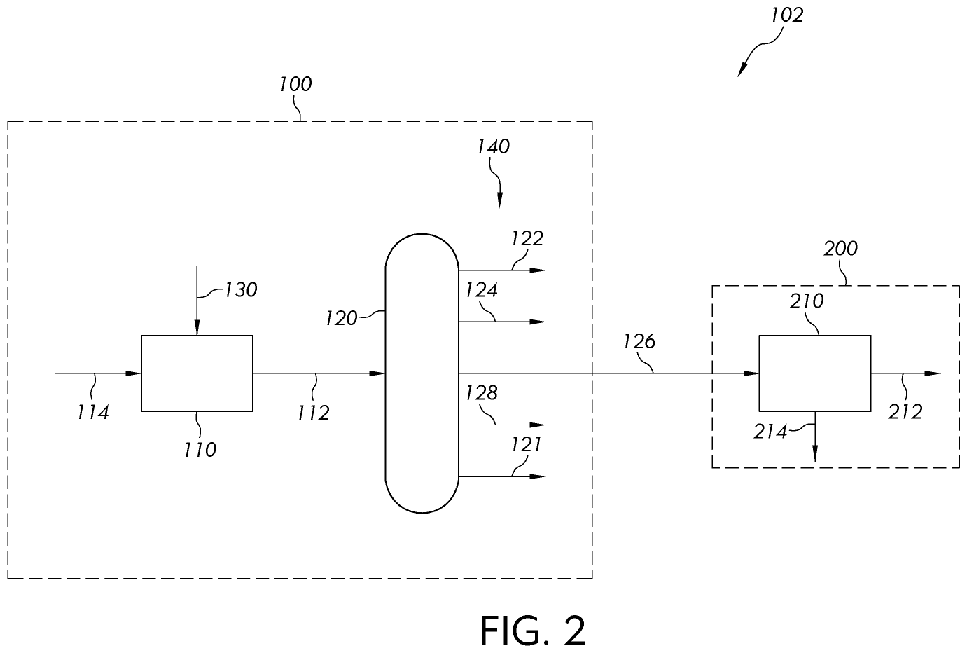

One or more embodiments of the present disclosure relate to methods for transporting hydrogen gas from one geographic region to another. In general, these methods are described herein in the context of one or more systems, shown in the drawings. The embodiments of FIGS. 1-5 are similar or identical in many ways, respectively, but include differences as described herein. Description of the embodiments of FIGS. 1-5 may generally apply to the embodiments of the other figures, as would be understood by those skilled in the art. For example, concepts disclosed herein applicable to FIG. 1 may be equally applicable to FIG. 2, and vice versa, even if not explicitly stated as such herein.

Now referring to FIG. 1, a system for transporting hydrogen 101 is depicted. The system for transporting hydrogen 101 may include at least a first chemical processing facility 100 and a second chemical processing facility 200, where the first chemical processing facility 100 and the second chemical processing facility 200 are in different geographic locations, such as separated by a distance of at least 100 km, as described herein. In general, a single chemical processing facility, such as the first chemical processing facility 100 and a second chemical processing facility 200, may be a processing facility that is only locally integrated with other processing facilities, and generally refers to an integrated complex capable of transforming its respective chemical feedstock into its respective products. For example, a single chemical processing facility may be under the control of a single entity, such as a company. For example, the first chemical processing facility 100 and a second chemical processing facility 200 may be in different geographic regions, such as different states, countries, counties, provinces, continents, etc.

The first chemical processing facility 100 and the second chemical processing facility 200 may be separate from one another and in different geographic regions. For example, the first chemical processing facility 100 and the second chemical processing facility 200 may be at least 100 km apart from one another, such as at least 200 km apart from one another, at least 500 km apart from one another, or at least 1000 km apart from one another.

The physical distance between the first chemical processing facility 100 and the second chemical processing facility 200 may make conventional transportation of hydrogen gas between the first chemical processing facility 100 and the second chemical processing facility 200 difficult and/or costly. Use of the present methods and systems may allow for cheaper and/or more efficient transport of hydrogen between the first chemical processing facility 100 and the second chemical processing facility 200, thereby allowing an operator to take advantage of cheaper and/or “sustainable” sources of hydrogen gas available near the first chemical processing facility 100. In some embodiments, the first chemical processing facility 100 and the second chemical processing facility 200 may be located at different latitudes, which may allow the operator to take advantage of variations in energy production, such as the increased production of electricity of a given solar panel when placed closer to the equator or change in seasonality.

Still referring to FIG. 1, the first chemical processing facility 100 may comprise at least a first reactor 110 and the second chemical processing facility 200 may comprise at least a dehydrogenation unit 210. These system components and their various arrangements will be described in detail herein.

According to one or more embodiments, at the first chemical processing facility 100, a carbon monoxide feed 114 and a hydrogen gas feed 130 may be passed to the first reactor 110 to form mixed hydrocarbon product 112. The carbon monoxide feed 114 and the hydrogen gas feed 130 may be combined before entering the first reactor 110 or may be combined therein. Thus, hydrogen gas from the hydrogen gas feed 130 may be embodied in hydrocarbons in the mixed hydrocarbon product 112. This mixed hydrocarbon product 112 may be easier to transport than hydrogen gas.

In embodiments, the hydrogen gas feed 130 may comprise hydrogen gas. In embodiments, the hydrogen gas feed 130 may comprise at least 50 wt. %, at least 80 wt. %, at least 90 wt. %, at least 95 wt. %, or at least 99 wt. % of hydrogen gas, on the basis of the total weight of hydrogen gas feed 130. As described in more detail herein, the hydrogen gas in the hydrogen gas feed 130 may be produced from “sustainable” processes (e.g., processes which do not release carbon dioxide), from hydrocarbons (e.g., partial oxidation or partial steam reforming), or a combination thereof. In some embodiments, the hydrogen gas feed 130, or portions thereof, may be a byproduct of forming the carbon monoxide feed 114.

In embodiments, the carbon monoxide feed 114 may comprise carbon monoxide. In embodiments, the carbon monoxide feed 114 may comprise may comprise at least 50 wt. %, at least 80 wt. %, at least 90 wt. %, at least 95 wt. %, or at least 99 wt. % of carbon monoxide, on the basis of the total weight of carbon monoxide feed 114. Carbon monoxide may be produced from the partial oxidation and/or steam reforming of hydrocarbons in a hydrocarbon gasification reactor (not shown in the figures). Thus, the system for transporting hydrogen 101 may include a hydrocarbon gasification reactor (not shown in the figures) upstream of the first reactor 110, in which hydrocarbons are partially oxidized and/or steam reformed to produce a mixture of carbon monoxide and hydrogen gas (also referred to as syngas). The syngas may include other chemicals such as carbon dioxide. The hydrocarbons fed to the hydrocarbon gasification reactor may include liquid hydrocarbons, such as crude oil or crude oil distillates; gaseous hydrocarbons such as natural gas; and/or solid hydrocarbons such as coal. This wide variety of hydrocarbon feeds may decrease the operational costs of the system. A portion of the hydrogen gas in hydrogen gas feed 130 may be produced by this hydrocarbon gasification reactor, thus providing inexpensive and readily available hydrogen gas to the first reactor 110. It should be understood that while the hydrogen gas feed 130 and the carbon monoxide feed 114 are shown as separate from one another in the figures, in some embodiments, a single feed comprising the carbon monoxide and the hydrogen gas may be used.

Still referring to FIG. 1, in the first reactor 110, the carbon monoxide and the hydrogen gas may undergo a Fischer-Tropsch reaction to form the mixed hydrocarbon product 112. Generally, a Fischer-Tropsch reaction combines carbon monoxide and hydrogen to form hydrocarbons. While a Fischer-Tropsch reaction produces a variety of hydrocarbon molecules depending on the operational conditions utilized, in some embodiments, the mixture of hydrocarbon molecules produced may comprise alkanes formed according to equation (1):

$$( 2 n + 1 ) {H}_{2} + n CO \to {C}_{n} {H}_{2 n + 2} + n {H}_{2} O \qquad ( 1 )$$

In a Fischer-Tropsch reaction, the carbon monoxide and the hydrogen gas may contact a Fischer-Tropsch catalyst under suitable conditions for Fischer-Tropsch reactions. The Fischer-Tropsch catalyst refers to any substance which increases the rate of a Fischer-Tropsch reaction, such as a catalyst designed to promote Fischer-Tropsch reactions. Contemplated Fischer-Tropsch catalysts include metals, such as Fe, Co, or Co—Fe. The metals may be supported, such as on an alumina support. The Fischer-Tropsch reaction conditions may include a temperature of from 150° C. to 300° C., such as from 150° C. to 175° C., from 175° C. to 200° C., from 200° C. to 225° C., from 225° C. to 250° C., from 250° C. to 275° C., from 275° C. to 300° C., or any combination of these ranges. The Fischer-Tropsch reaction conditions may include a reaction pressure of from 1 bar to 50 bar, such as from 1 bar to 5 bar, from 5 bar to 10 bar, from 10 bar to 15 bar, from 15 bar to 20 bar, from 20 bar to 30 bar, from 30 bar to 40 bar, from 40 bar to 50 bar, or any combination of these ranges. The first reactor 110 may be any reactor type suited for contacting gaseous components with a catalyst. For example, the first reactor 110 may be a fixed bed reactor, a moving bed reactor, or a fluidized bed reactor.

Contacting the carbon monoxide and the hydrogen gas with the Fischer-Tropsch catalyst may produce the mixed hydrocarbon product 112. The mixed hydrocarbon product 112 may comprise a mixture of hydrocarbon molecules, such as linear alkanes, branched alkanes, and linear olefins. In embodiments, at least 80 wt. %, at least 90 wt. %, or at least 99 wt. % of the hydrocarbon molecules in the mixed hydrocarbon product 112 may comprise from 1 carbon atom to 70 carbon atoms. In embodiments, at least 50 wt. %, at least 75 wt. %, at least 80 wt. %, at least 85 wt. %, at least 90 wt. %, or at least 95 wt. % of the hydrocarbons in the mixed hydrocarbon product 112 may be the combined weight of linear alkanes, branched alkanes, and linear olefins. In embodiments, at least 30 wt. %, at least 50 wt. %, at least 75 wt. %, at least 80 wt. %, at least 85 wt. %, at least 90 wt. %, or at least 95 wt. % of the hydrocarbons in the mixed hydrocarbon product 112 may be the alkanes. Generally, when the concentration of alkanes in the mixed hydrocarbon product 112 the highest, the hydrogen carrying capacity (i.e., the amount of hydrogen which can be stored in the mixed hydrocarbon product 112 and released to form the hydrogen gas product 212, per mass of the mixed hydrocarbon product 112) will be the highest. In some embodiments, the mixed hydrocarbon product 112 may be liquid at ambient temperature and pressure (e.g., 20° C. and 1 atmosphere). In some embodiments, only a portion of the mixed hydrocarbon product 112 may be liquid at ambient temperature and pressure.

In embodiments not shown in the figures, the mixed hydrocarbon product 112 may be hydrogenated at the first chemical processing facility 100 in a hydrogenation unit to convert olefins in the mixed hydrocarbon product 112 to alkanes. Hydrogenation refers to a chemical reaction in which the number of hydrogen atoms or the concentration of hydrogen atoms in a stream is increased. Hydrogenation reactions may include hydrotreating reactions and hydrocracking reactions. Generally, when a significant portion of the mixed hydrocarbon product 112 comprises olefins, it may be beneficial to hydrogenate the olefins to increase the hydrogen carrying capacity of the mixed hydrocarbon product 112. The hydrogenation unit may include a hydrogenation catalyst, where the hydrocarbons and other chemicals present in the hydrogenation unit may be contacted with the hydrogenation catalyst in the presence of hydrogen gas. The hydrogenation catalyst may be a specialized hydrogenation catalyst, a hydrotreating catalyst, or a hydrocracking catalyst such as, cobalt-molybdenum (Co—Mo), nickel-molybdenum (Ni—Mo), nickel-tungsten (Ni—W), and/or noble metal catalysts. The hydrogenation catalyst may include a solid acid material, such as amorphous silica alumina or zeolites. In embodiments, the catalyst may be supported by alumina. The hydrogenation unit may be operated at a reaction temperature from 50° C. to 700° C., such as from 50° C. to 100° C., from 100° C. to 200° C., from 200° C. to 300° C., from 300° C. to 400° C., from 400° C. to 500° C., from 500° C. to 600° C., from 600 to 700° C. or any combination of these ranges. The hydrogenation unit may be operated at a pressure of from 10 bar to 300 bar, such as from 10 bar to 250 bar, from 10 bar to 200 bar, from 10 bar to 150 bar, from 15 bar to 300 bar, from 15 bar to 200 bar, from 15 bar to 150 bar, or any subset thereof. The hydrogenation unit may be operated at a liquid hourly space velocity (“LHSV”) of from 0.5 h⁻¹ to 5 h⁻¹, such as from 0.5 h⁻¹ to 1 h⁻¹, from 1 h⁻¹ to 2 h⁻¹, from 2 h⁻¹ to 3 h⁻¹, from 3 h⁻¹ to h⁻¹, from 4 h⁻¹ to 5 h⁻¹, or any combination of these ranges.

Still referring to FIG. 1, the mixed hydrocarbon product 112, or a portion thereof, may be transported from the first chemical processing facility 100 to the second chemical processing facility 200. Transporting the mixed hydrocarbon product 112, or a portion thereof may transport hydrogen, in the form of hydrogen atoms covalently bonded to the hydrocarbons, from the first chemical processing facility 100 to the second chemical processing facility 200. Transporting may refer to the process of physically moving hydrocarbons, and to the process of preparing the hydrocarbons to be physically moved, from the first chemical processing facility 100 to the second chemical processing facility 200, and to the storage of hydrocarbons before, during, or after physical movement of the hydrocarbons. In embodiments, transporting the mixed hydrocarbon product 112, or a portion thereof, may comprise transporting the mixed hydrocarbon product 112, or a portion thereof, from the first chemical processing facility 100 to the second chemical processing facility 200 by tanker truck, train, ship, pipeline, or the like. In embodiments, the hydrocarbons may be transported from the first chemical processing facility 100 to the second chemical processing facility 200 by tanker truck, train, and/or ship. The transportation process may utilize pre-existing crude oil and petroleum products transportation infrastructure. A time of at least 2 weeks, such as at least 1 month, at least 2 months, or at least 6 months, may pass between forming the mixed hydrocarbon product 112 and dehydrogenating the mixed hydrocarbon product 112. The transportation step may include storing the hydrocarbons at the first chemical processing facility 100, the second chemical processing facility 200, at an intermediate storage or processing facility, or in the transportation vessel itself. The temporal difference between the Fischer-Tropsch reaction and dehydrogenation may allow the operator to store intermittent electricity in the form of hydrogen for use during times of higher demand, such as storing summer solar power for winter.

In embodiments, at least 30 wt. % of the mixed hydrocarbon product 112 may be transported from the first chemical processing facility 100 to the second chemical processing facility 200. In embodiments, at least 50 wt. %, at least 60 wt. %, at least 75 wt. %, at least 80 wt. %, or at least 90 wt. % of the mixed hydrocarbon product 112 may be transported from the first chemical processing facility 100 to the second chemical processing facility 200. In embodiments, the portion of the mixed hydrocarbon product 112 that is transported does not undergo separation by boiling point at the first chemical processing facility 100. For example, some of the mixed hydrocarbon product 112 may be diverted whole to other uses or other hydrocarbon processing facilities but the portion of mixed hydrocarbon product 112 which is transported may still have the same composition as when it exited first reactor 110.

In embodiments, at least 30 wt. % of the mixed hydrocarbon product 112 may be dehydrogenated at the second chemical processing facility 200. In embodiments, the portion of the mixed hydrocarbon product 112 that is transported does not undergo separation by boiling point before being dehydrogenated at the second chemical processing facility 200. In embodiments, at least 50 wt. %, at least 60 wt. %, at least 75 wt. %, at least 80 wt. %, or at least 90 wt. % of the mixed hydrocarbon product 112 may be dehydrogenated at the second chemical processing facility 200.

Still referring to FIG. 1, at the second chemical processing facility 200, the mixed hydrocarbon product 112, or portion thereof, that was transported, may be passed to dehydrogenation unit 210 to form dehydrogenated effluent 214 and hydrogen gas product 212. The dehydrogenation unit 210 may be any process unit capable of removing hydrogen atoms from a hydrogen molecule to form hydrogen gas. In embodiments, the dehydrogenation unit 210 may be a process unit of an oil refinery or a petrochemical plant, such as a petrochemical plant which was already dehydrogenating a hydrocarbon feed. Although not shown in the figures, the mixed hydrocarbon product 112, or portion thereof, that was transported, may be dehydrogenated in the dehydrogenation unit 210 at the same time as an additional hydrocarbon feed (e.g., hydrotreated naphtha), thereby reducing capital costs. Suitable process units for use as dehydrogenation unit 210 include, for example, propane dehydrogenation units, butane dehydrogenation units, steam crackers, catalytic reformers, aromatization units, and the like.

The dehydrogenation unit 210 may include a catalyst, where the hydrocarbons and other chemicals in the dehydrogenation unit 210 may be contacted with the catalyst to dehydrogenate the hydrocarbons and release hydrogen gas. Contemplated catalysts may comprise iron (e.g., iron (III) oxide), potassium oxide, potassium chloride, noble metal (e.g., Pt or Re), Pt—Sn, Pt—Ga, Cr. Contemplated catalysts may include a promoter, such as Na or K. Contemplated catalysts include those supported on an alumina base, silica base, a silica-alumina base, a zirconia base, a ZnAl₂O₃ base, a CaAl₂O₃ base, or a combination thereof. The dehydrogenation unit 210 may operate at a reaction temperature of from of from 50° C. to 700° C., such as from 50° C. to 100° C., from 100° C. to 200° C., from 200° C. to 300° C., from 300° C. to 400° C., from 400° C. to 500° C., from 500° C. to 600° C., from 400° C. to 600° C., or any combination of these ranges; a reaction pressure of from 1 bar to 50 bar, such as from 1 bar to 5 bar, from 5 bar to 10 bar, from 10 bar to 20 bar, from 20 bar to 30 bar, from 30 bar to 40 bar, from 40 bar to 50 bar, or any combination of these ranges; and a liquid hourly space velocity (“LHSV”) of from 0.5 h⁻¹ to 5 h⁻¹, such as from 0.5 h⁻¹ to 1 h⁻¹, from 1 h⁻¹ to 2 h⁻¹, from 2 h⁻¹ to 3 h⁻¹, from 3 h⁻¹ to 4 h⁻¹, from 4 h⁻¹ to 5 h⁻¹, or any combination of these ranges.

As is shown in FIG. 1, the dehydrogenation unit 210 may produce a dehydrogenated effluent 214 and a hydrogen gas product 212. Although dehydrogenated effluent 214 and hydrogen gas product 212 are shown leaving the dehydrogenation unit 210 as separate streams, they may leave dehydrogenation unit 210 as a single stream with hydrogen gas entrained in the hydrocarbons. The hydrogen gas may be separated from the hydrocarbons in a liquid-gas separator (not shown in the figures) downstream of the dehydrogenation unit 210 to produce the dehydrogenated effluent 214 and the hydrogen gas product 212.

The dehydrogenated effluent 214 may comprise the dehydrogenated hydrocarbons initially passed to the dehydrogenation unit 210. In embodiments, the dehydrogenated effluent 214 may have a lesser ratio of hydrogen to carbon than the mixed hydrocarbon product 112, or the portions thereof which are fed to the dehydrogenation unit 210. For example, the degree of saturation of the hydrocarbons in the dehydrogenated effluent 214 may be lower than the degree of saturation in the mixed hydrocarbon product 112. Thus, hydrogen atoms from the mixed hydrocarbon product 112 may form the hydrogen gas product 212. The dehydrogenated effluent 214 may comprise valuable petrochemical products, which maybe further converted or sold at the second chemical processing facility 200.

The hydrogen gas product 212 may comprise hydrogen gas. In embodiments, hydrogen gas product 212 may comprise hydrogen gas, such as at least 60 wt. %, at least 80 wt. %, at least 90 wt. %, at least 95 wt. %, at least 99 wt. %, at least 99.9 wt. %, at least 99.99 wt. %, or 99.999 wt. % of hydrogen gas, on the basis of the total weight of the hydrogen gas product 212. Additionally, the hydrogen gas product 212 may include contaminants, such as carbon monoxide and carbon dioxide. One practical and growing application for hydrogen gas is for use in fuel cells. Generally, low temperature fuel cells use precious metal catalysts which are susceptible to poisoning by CO in their hydrogen fuels. To produce fuels suitable for such uses, it may be desirable to produce a hydrogen gas product 212 with a minimum amount of contaminants, such as CO. Accordingly, the hydrogen gas product 212 may be subjected to a further purification process (not shown in the figures), such as pressure swing adsorption, electrochemical pumping, or cryogenic separation.

Referring now to FIG. 2, another system for transporting hydrogen 102 is depicted. The system for transporting hydrogen 102 may be similar or identical to the system for transporting hydrogen 101 of FIG. 1, except where described otherwise. In particular, in the system for transporting hydrogen 102, the mixed hydrocarbon product 112 may be passed to a separation unit 120 to separate the mixed hydrocarbon product 112 into one or more hydrocarbon cuts 140. One or more of these hydrocarbon cuts 140 (each of which are a portion of the mixed hydrocarbon product 112) may then be transported to the additional hydrocarbon processing facilities, such as the second chemical processing facility 200, thereby transporting the hydrogen atoms between the hydrocarbon processing facilities.

As used in this disclosure, a “separation unit” refers to any separation device or system of separation devices that at least partially separates one or more chemicals that are mixed in a process stream from one another. For example, a separation unit may selectively separate differing chemical species, phases, or sized material from one another, forming one or more chemical fractions. Examples of separation units include, without limitation, distillation columns, flash drums, knock-out drums, knock-out pots, centrifuges, cyclones, filtration devices, traps, scrubbers, expansion devices, membranes, solvent extraction devices, and the like. It should be understood that separation processes described in this disclosure may not completely separate all of one chemical constituent from all of another chemical constituent. It should be understood that the separation processes described in this disclosure “at least partially” separate different chemical components from one another, and that even if not explicitly stated, it should be understood that separation may include only partial separation. The separation unit 120 may be any suitable separation unit, such as, and without limitation, a series of flash vessels or a fractionator/distillation column that separates feedstock based on the boiling point, to remove the separated compounds.

The separation process will now be described in more specificity. As depicted in FIG. 2, the mixed hydrocarbon product 112 may be separated into two or more hydrocarbon cuts 140 in separation unit 120. In embodiments, the mixed hydrocarbon product 112 may be separated into at least a first hydrocarbon cut 126 and a second hydrocarbon cut 124. In embodiments, the first hydrocarbon cut 126 may comprise hydrocarbons having an initial boiling point (“IBP”) of from 30° C. to 100° C., such as from 30° C. to 50° C., from 50° C. to 60° C., from 60° C. to 70° C., from 70° C. to 80° C., from 80° C. to 90° C., from 90° C. to 100° C., or any combination of these ranges. The first hydrocarbon cut 126 may comprise hydrocarbons having a final boiling point (“FBP”) of from 180° C. to 220° C., such as from 180° C. to 190° C., from 190° C. to 200° C., from 200° C. to 210° C., from 210° C. to 220° C., from 195° C. to 205° C., or combination of these ranges. In some embodiments, the first hydrocarbon cut 126 may be liquid at ambient temperature and pressure (e.g., 20° C. and 1 atmosphere). Thus, enabling easier transport of the hydrogen atoms in the first hydrocarbon cut 126.

In embodiments, the second hydrocarbon cut 124 may comprise hydrocarbons boiling at a lower temperature than the hydrocarbons of the first hydrocarbon cut 126. In embodiments, the second hydrocarbon cut 124 may comprise C1-C4 hydrocarbons. In embodiments, the second hydrocarbon cut 124 may comprise at least 50 wt. %, at least 75 wt. %, at least 85 wt. %, at least 90 wt. %, at least 95 wt. %, or at least 99 wt. % of C1-C4 hydrocarbons. In embodiments, the second hydrocarbon cut 124 may comprise propane, butane, or both. In embodiments, the second hydrocarbon cut 124 may comprise at least 50 wt. %, at least 75 wt. %, at least 85 wt. %, at least 90 wt. %, at least 95 wt. %, or at least 99 wt. % of propane, butane, or both. Although C1-C4 hydrocarbons are not a liquid at ambient temperature and pressure, C1-C4 hydrocarbons are still easier to transport than hydrogen gas as the molecules are larger and they are less energy intensive to liquefy.

In embodiments, in addition to the first hydrocarbon cut 126 and the second hydrocarbon cut 124, the mixed hydrocarbon product 112 may be separated into a third hydrocarbon cut 128, a fourth hydrocarbon cut 122, and a fifth hydrocarbon cut 121. The third hydrocarbon cut 128 may comprise hydrocarbons having a boiling point range between the boiling point range of the first hydrocarbon cut 126 and the boiling point range of the fifth hydrocarbon cut 121. The fourth hydrocarbon cut 122 may comprise hydrocarbons having a boiling point range less than the boiling point range of the second hydrocarbon cut 124. The fifth hydrocarbon cut 121 may be the heaviest of the hydrocarbon cuts 140 and may have a boiling point range greater than that of the third hydrocarbon cut 128. Generally, the fifth hydrocarbon cut 121 may be difficult to dehydrogenate and thus, the fifth hydrocarbon cut 121 is often not transported to the additional hydrocarbon processing facilities, such as the second chemical processing facility 200.

Still referring to FIG. 2, transporting at least a portion of the mixed hydrocarbon product 112 from the first chemical processing facility 100 to the second chemical processing facility 200 may comprise transporting one or more of the hydrocarbon cuts 140 from the first chemical processing facility 100 to the second chemical processing facility 200. In embodiments, transporting at least a portion of the mixed hydrocarbon product 112 from the first chemical processing facility 100 to the second chemical processing facility 200 may comprise transporting first hydrocarbon cut 126 from the first chemical processing facility 100 to the second chemical processing facility 200.

At the second chemical processing facility 200, dehydrogenating at least a portion of mixed hydrocarbon product 112 may comprise dehydrogenating the hydrocarbon cuts 140 which are transported from the first chemical processing facility 100 to the second chemical processing facility 200. In embodiments, the first hydrocarbon cut 126 may be dehydrogenated in the dehydrogenation unit 210 at the second chemical processing facility 200.

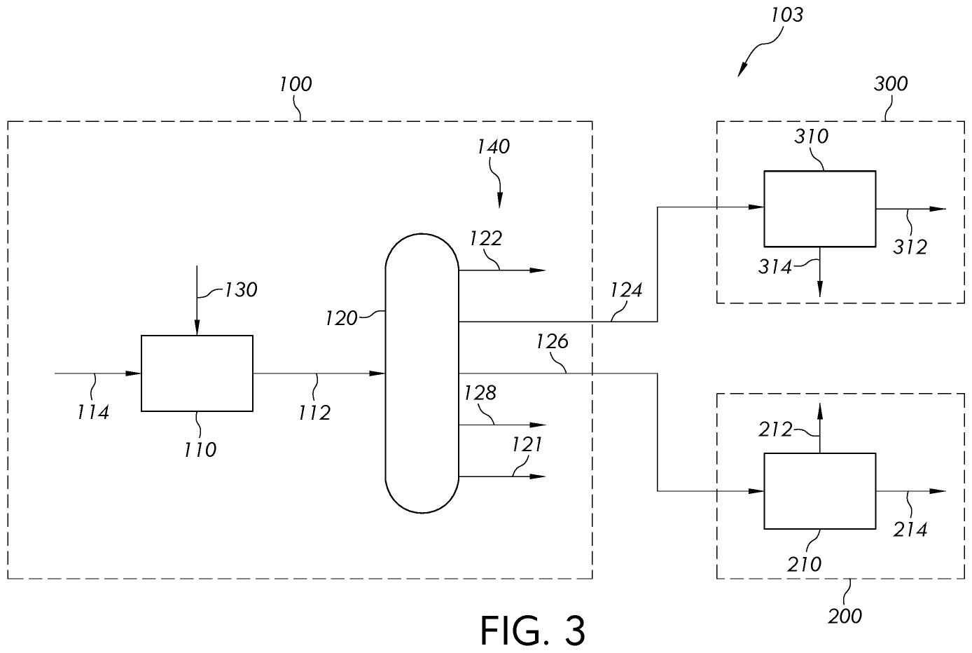

Referring now to FIG. 3, another system for transporting hydrogen 103 is depicted. The system for transporting hydrogen 103 may be similar or identical to the system for transporting hydrogen 102 of FIG. 2, except where described otherwise. In the system for transporting hydrogen 103, one or more of the hydrocarbon cuts 140 may be transported to additional chemical processing facilities. In some embodiments, the second hydrocarbon cut 124 may be transported to the third chemical processing facility 300 and dehydrogenated therein.

The third chemical processing facility 300 may comprise any new or conventional chemical processing facility capable of dehydrogenating one or more hydrocarbon cuts 140 (e.g., the second hydrocarbon cut 124). For example, the third chemical processing facility 300 may be an oil refinery or a petrochemical plant. The third chemical processing facility may be at least 100 km, such as at least 200 km, at least 500 km, or at least 100 km from the first chemical processing facility 100. The third chemical processing facility 300 may comprise any of the dehydrogenation units discussed herein in reference to the second chemical processing facility 200 (e.g., a propane dehydrogenation unit, a butane dehydrogenation unit, a steam cracker, an aromatization unit, or a catalytic reformer). The dehydrogenation unit 310 may be any of the dehydrogenation units described herein for dehydrogenation unit 210 and may be operated under substantially similar operating conditions to dehydrogenation unit 210. The dehydrogenated effluent 314 may be substantially similar to dehydrogenated effluent 214. The hydrogen gas product 312 may be substantially similar to hydrogen gas product 212.

In some specific embodiments, the second hydrocarbon cut 124 may comprise propane, butane, or both, such as at least 50 wt. %, at least 75 wt. %, at least 85 wt. %, at least 95 wt. %, or at least 99 wt. % of propane, butane, or both, on the basis of the total weight of second hydrocarbon cut 124. In such embodiments, the dehydrogenation unit 310 may be a propane dehydrogenation unit, a butane dehydrogenation unit, or both. In a propane dehydrogenation unit, the propane from the second hydrocarbon cut 124 may be converted to propylene. In a butane dehydrogenation unit, the butane from the second hydrocarbon cut 124 may be converted to butylene. In embodiments, the dehydrogenated effluent 314 may comprise propylene, butylene, or both, such as at least 80 wt. %, at least 90 wt. %, at least 95 wt. %, at least 99 wt. %, at least 99.9 wt. %, or even at least 99.99 wt. % of propylene, butylene, or both, on the basis of the total weight of dehydrogenated effluent 314.

Still referring to FIG. 3, in embodiments, at least 30 wt. % of the mixed hydrocarbon product 112 may be transported from the first chemical processing facility 100 to the additional chemical processing facilities, such as the second chemical processing facility 200 and the third chemical processing facility 300. In embodiments, at least 50 wt. %, at least 60 wt. %, at least 75 wt. %, at least 80 wt. %, or at least 90 wt. % of the mixed hydrocarbon product 112 may be transported from the first chemical processing facility 100 to the additional chemical processing facilities.

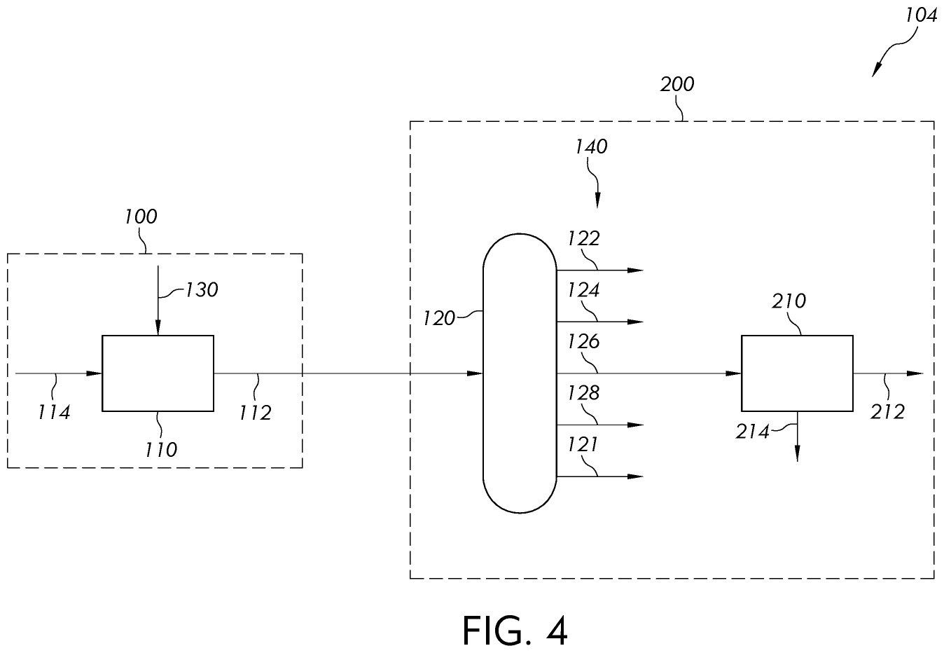

Referring now to FIG. 4, another system for transporting hydrogen 104 is depicted. The system for transporting hydrogen 104 may be similar or identical to the system for transporting hydrogen 102 of FIG. 2, except where described otherwise. In particular, in the system for transporting hydrogen 104, the mixed hydrocarbon product 112, or a portion thereof, may be transported to the second chemical processing facility 200 and separated at the second chemical processing facility 200 to form the hydrocarbon cuts 140. As described for system for transporting hydrogen 102, one or more of the hydrocarbon cuts 140 may then be dehydrogenated at the second chemical processing facility 200 to form the hydrogen gas product 212. In embodiments, the portion of the mixed hydrocarbon product 112 that is transported does not undergo separation by boiling point at the first chemical processing facility 100. In other embodiments not shown in the figure, the portion of the mixed hydrocarbon product 112 is that is transported may be separated by boiling point to form a hydrocarbon cut 140, that hydrocarbon cut 140 may be transported to the second chemical processing facility 200, where the hydrocarbon cut 140 that is transported may be further separated to form additional hydrocarbon cuts, at least one of which may then be dehydrogenated to form the hydrogen gas product.

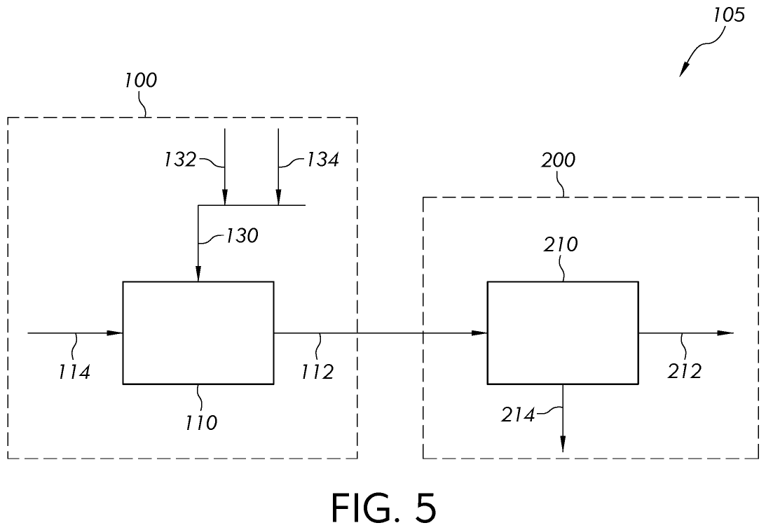

Referring now to FIG. 5, another system for transporting hydrogen 105 is depicted. The system for transporting hydrogen 105 may be similar or identical to the system for transporting hydrogen 101 of FIG. 1, except where described otherwise. In particular, in the system for transporting hydrogen 105, the hydrogen gas feed 130 may be formed from a first hydrogen gas portion 132 produced by a method with no direct carbon emissions to the atmosphere and a second hydrogen gas portion 134 produced by a method with direct carbon emissions to the atmosphere. Direct carbon emissions generally refers to carbon, such as in the form of carbon dioxide, that is emitted to the environment (i.e., not sequestered) in a process that produces hydrogen, such as from the release of carbon dioxide from fossil fuels during hydrogen production. Generally, when the ratio of the first hydrogen portion 132 and the second hydrogen portion 134 is optimized, it may be possible to attribute most or all of the hydrogen produced at the chemical processing facilities other than the first chemical processing facility 100 (e.g., the second chemical processing facility 200 and the third chemical processing facility 300) to the first hydrogen portion 132, as will be described in more detail herein.

Hydrogen may be produced by a wide variety of methods and resources. Based on environmental sustainability, some hydrogen may be more valuable based on its origin and production means. For example, hydrogen that is produced with little or no carbon emission may be more valuable in the marketplace. These include the first, second, and third hydrogen production methods described herein below, which produce hydrogen with no direct carbon emissions, as well as hydrogen that is naturally occurring (sometimes referred to as “white hydrogen” in industry).

A first hydrogen generation technique includes a water electrolysis process by employing renewable electricity (sometimes referred to as “green hydrogen” in industry). Generally, there is no carbon dioxide emissions during this production technique. Renewable electricity may be generated from, without limitation, wind, solar, geothermal, hydroelectric, marine, biomass, and other forms of electricity produced from renewable resources that do not contribute to carbon emissions.

In embodiments, the first hydrogen portion may be produced by water electrolysis. However, as described herein, other methods of producing the first hydrogen portion that do not form direct carbon emissions are also contemplated. According to one or more embodiments, water may be passed to an electrolysis cell (not depicted in the figures), where a source of electricity is used to produce the first hydrogen gas portion 132. The electrolysis cell may produce the first hydrogen gas portion 132 from water and electricity through the process of electrolysis. Generally, the electrolysis of water refers to the decomposition of water into oxygen gas and hydrogen gas by passing an electric current through the water. Generally, electrochemical cells configured for the production of hydrogen have two electrodes, a cathode and an anode. The electrodes are immersed in the water and externally connected with a power supply. At a critical voltage, hydrogen and oxygen are produced. The reactions proceed as follows:

2H⁺(aq)+2e⁻→H₂(g) (1)

4OH⁻(aq)→2H₂O+O₂(g)+4e⁻ (2)

The electrolysis cell may be characterized by the chemistry of the electrolyte. For example, the electrolysis cell may be an alkaline electrolysis cell comprising a porous separator and an alkaline electrolyte or a proton exchange membrane (“PEM”) electrolysis cell comprising an acidic membrane separator which also functions as an acidic electrolyte, or a solid oxide electrolysis cell comprising a solid oxide electrolyte (e.g., yttrium-stabilized-zirconia). The anodes and the cathodes may be unipolar or bipolar. When the electrodes are unipolar, each electrode functions as a terminal electrode. Bipolar electrolyzers may include many cells operating in series. Each electrode in the bipolar electrolyzer, with the exception of the two terminal electrodes, may function as a cathode on one side of the electrode and an anode at the other side of the electrode.

The electrolysis cell may be operated at a temperature in the range from 10° C. to 99° C., such as from 10° C. to 20° C., from 20° C. to 30° C., from 30° C. to 40° C., from 40° C. to 50° C., from 50° C. to 60° C., from 60° C. to 70° C., from 70° C. to 80° C., from 80° C. to 90° C., from 90° C. to 99° C., or any combination of these ranges. In embodiments, the electrolysis cell 150 may be an alkaline or PEM electrolysis cell and the operating temperature may be in the range from 10° C. to 99° C. In embodiments, the electrolysis cell may be a solid oxide electrolysis cell operated at a temperature in the range from 500° C. to 900° C. The electrolysis cell 150 may be operated at a hydrogen pressure of from 1 bar to 500 bar, such as from 1 bar to 10 bar, from 10 bar to 50 bar, from 50 bar to 100 bar, from 100 bar to 200 bar, from 200 bar to 300 bar, from 300 bar to 400 bar, from 400 bar to 500 bar, or any combination of these ranges.

The source of electricity to the electrolysis cell may be electricity produced from energy provided by a non-hydrocarbon source. Generally, the use of electricity produced from energy provided by a non-hydrocarbon source means that first hydrogen gas portion produced in the electrolysis cell may be referred to as “sustainable” hydrogen and may thus be more valuable than other types of hydrogen. Suitable sources of electricity may include, by way of example but not limitation, solar energy, wind energy, hydroelectric energy, geothermal energy, tidal energy, or a combination thereof.

A second hydrogen generation technique includes producing hydrogen by utilization of fossil fuels, but all of the generated carbon dioxide is captured and sequestered, such as sequestered underground (sometimes referred to as “gray hydrogen” in industry). Since no carbon is emitted into the atmosphere, this is considered carbon neutral in industry.

A third hydrogen generation technique includes extracting energy from nuclear reactions, where in general carbon dioxide is not emitted. Several techniques are known for forming hydrogen from nuclear fission and/or fusion, including hydrogen that is made though using nuclear power and heat through combined chemo thermal electrolysis splitting of water (sometimes referred to as “purple hydrogen” in industry); hydrogen that is generated through electrolysis of water by using electricity from a nuclear power plant (also sometimes referred to as “purple hydrogen” in industry); and hydrogen that is produced through the high-temperature catalytic splitting of water using nuclear power thermal as an energy source. (sometimes referred to as “red hydrogen” in industry).

The second hydrogen gas portion 134 may comprise hydrogen produced by a method with direct emissions to the atmosphere, such as from a hydrocarbon source in the absence of carbon sequestration. These include, without limitation, hydrogen is produced from fossil fuel and commonly uses steam methane reforming (SMR) method (sometimes referred to as “gray hydrogen” in industry), hydrogen is produced from coal, usually by gasification (sometimes referred to as “black hydrogen” or “brown hydrogen” in industry). This hydrogen produced by a method with direct emissions to the atmosphere may be less valuable than other types of hydrogen and/or more readily available, especially at chemical processing facilities, such as oil refineries, where hydrogen produced by a method with direct emissions to the atmosphere may be a byproduct chemical already in production. Producing hydrogen from a hydrocarbon source may refer to converting the hydrocarbons to hydrogen and carbon dioxide (although other compounds such as carbon monoxide may also be generated). For example, hydrogen may be produced from a hydrocarbon source through partial oxidation gasification process and subsequent water-gas shift reactions. Generally, a partial oxidation gasification process converts hydrocarbons into a gaseous mixture often referred to as syngas (i.e., a mixture of carbon monoxide and hydrogen). The carbon monoxide is then combined with high temperature steam to convert the carbon monoxide in the syngas to hydrogen and carbon dioxide. However, other common processes such as catalytic reforming also produce hydrogen gas from hydrocarbons, this hydrogen gas may form a part of the second hydrogen gas portion 134.

In some embodiments, where both the first hydrogen gas portion 132 and the second hydrogen gas portion 134 are present, in order to produce “sustainable” hydrogen at the second chemical processing facility 200, sufficient hydrogen from the first hydrogen gas portion 132 may be supplied to the first reactor 110, such that the most or all of the hydrogen released into the hydrogen gas product 212 can be attributed to the first hydrogen gas portion 132. Attributing hydrogen to the first hydrogen gas portion means that, on an overall mass balance, it is possible that the hydrogen atoms present in the hydrogen gas product 212 could have been supplied by the first hydrogen gas portion 132. Attributing hydrogen to the first hydrogen gas portion 132 does not require tracking individual hydrogen atoms through their respective flows and chemical bonds, but rather that the number of hydrogen atoms released could have been provided by the first hydrogen gas portion 132, based on the amounts of the first hydrogen gas portion 132 supplied and the amounts of the hydrogen gas product 212 produced. In embodiments, on average, a mass flow rate of the first hydrogen gas portion 132 may be at least 90%, such as at least 95%, at least 99%, at least 100%, at least 105%, or at least 110% of a mass flow rate of the hydrogen gas product 212. Due to the delays in transportation and storage of the mixed hydrocarbon product 112, the system for transporting hydrogen 105 may only approach steady state over a relatively long period of time, such as months or years. Thus, the average mass flow rates should be calculated over a time period of at least 6 months, at least 1 year, at least 2 years, at least 3 years, or at least 5 years. For example, if over a time period of at least 6 months (such as at least 1 year, at least 2 years, at least 3 years, or at least 5 years) the 1000 kg of hydrogen were released into the hydrogen gas product 212, at least 900 kg (such as at least 950 kg, at least 990 kg, at least 1000 kg, at least 1050 kg, or at least 1100 kg) of hydrogen would be supplied to the first reactor 110 by the first hydrogen gas portion 132.

Numerous aspects are included in the present disclosure, including aspects 1-20.
Aspect 1. A method of transporting hydrogen, the method comprising: at a first chemical processing facility, passing carbon monoxide and hydrogen gas to a first reactor, wherein the carbon monoxide and the hydrogen gas undergo a Fischer-Tropsch reaction in the first reactor and form a mixed hydrocarbon product, wherein the mixed hydrocarbon product comprises linear alkanes; transporting at least a portion of the mixed hydrocarbon product from the first chemical processing facility to a second chemical processing facility, wherein the first chemical processing facility and the second chemical processing facility are separated by a distance of at least 100 km; and at the second chemical processing facility, dehydrogenating at least a portion of the mixed hydrocarbon product to form a hydrogen gas product.
Aspect 2. The method of aspect 1, wherein the mixed hydrocarbon product comprises at least 50 wt. % of alkanes, on the basis of the total weight of the mixed hydrocarbon product.
Aspect 3. The method of any one of aspects 1-2, wherein at least 30 wt. % of the mixed hydrocarbon product is transported to the second chemical processing facility.
Aspect 4. The method of any one of aspects 1-2, wherein at least 80 wt. % of the mixed hydrocarbon product is transported to the second chemical processing facility.
Aspect 5. The method of any one of aspects 1-4, further comprising, at the first chemical processing facility, passing the mixed hydrocarbon product to a separation unit to form one or more hydrocarbon cuts, wherein transporting at least a portion of the mixed hydrocarbon product comprises transporting one or more of the hydrocarbon cuts.
Aspect 6. The method of aspect 5, wherein at least one of the hydrocarbon cuts that is transported to the second chemical processing facility is a first hydrocarbon cut that has an initial boiling point of from 20° C. to 100° C. and a final boiling point of from 180° C. to 220° C.
Aspect 7. The method of aspect 6, wherein the first hydrocarbon cut is dehydrogenated at the second chemical processing facility in a steam cracker to form the hydrogen gas product and light olefins.
Aspect 8. The method of any one of aspects 1-7, wherein dehydrogenating the at least a portion of the mixed hydrocarbon product that is transported to the second chemical processing facility comprises steam cracking or catalytic reforming.
Aspect 9. The method of any one of aspects 5-8, further comprising: transporting at least one of the hydrocarbon cuts from the first chemical processing facility to a third chemical processing facility, wherein the first chemical processing facility and the third chemical processing facility are separated by a distance of at least 100 km; and at the third chemical processing facility, dehydrogenating the one or more hydrocarbon cuts that are transported to the third chemical processing facility to form another hydrogen gas product.
Aspect 10. The method of aspect 9, wherein: at least one of the hydrocarbon cuts that is transported to the third chemical processing facility is a second hydrocarbon cut; and the second hydrocarbon cut comprises at least 50 wt. % of a combined weight of propane and butane, on the basis of the total weight of the second hydrocarbon cut.
Aspect 11. The method of any one of aspects 1-10, wherein transporting the at least a portion of the mixed hydrocarbon product that is transported from the first chemical processing facility to the second chemical processing facility comprises moving the at least a portion of the mixed hydrocarbon product by truck, train, or ship.
Aspect 12. The method of any one of aspects 1-11, wherein the at least a portion of the mixed hydrocarbon product that is transported from the first chemical processing facility to the second chemical processing facility is a liquid at a temperature of 20° C. and a pressure of 1 atmosphere.
Aspect 13. The method of any one of aspects 1-12, wherein: in the first reactor, the hydrogen and carbon monoxide are contacted with a Fischer-Tropsch catalyst comprising Fe, Co, or both; and the first reactor is operated at a reaction temperature of from 150° C. to 300° C. and a reaction pressure of from 1 bar to 50 bar.
Aspect 14. The method of any one of aspects 1-13, wherein the first reactor is a fixed-bed reactor or a fluidized bed reactor.
Aspect 15. The method of any one of aspects 1-14, wherein: the mixed hydrocarbon product exiting the first reactor comprises olefin compounds; and the method further comprises, at the first chemical processing facility, hydrogenating at least a portion of the mixed hydrocarbon product that exits the first reactor upstream of transporting the least a portion of the mixed hydrocarbon product to the second chemical processing facility.
Aspect 16. The method of any one of aspects 1-15, further comprising producing the carbon monoxide by partial oxidation of a hydrocarbon feed.
Aspect 17. The method of any one of aspects 1-16, wherein the portion of the mixed hydrocarbon product that is transported from the first chemical processing facility to the second chemical processing facility does not undergo separation by boiling point at the first chemical processing facility.
Aspect 18. The method of any one of aspects 1-17, further comprising, at the second chemical processing facility, passing the at least a portion of the mixed hydrocarbon product to a separator to form one or more hydrocarbon cuts, wherein one or more of the hydrocarbon cuts are dehydrogenated.
Aspect 19. The method of any one of aspects 1-18, wherein: a first hydrogen gas portion is hydrogen produced by a method with no direct carbon emissions to the atmosphere; a second hydrogen gas portion is hydrogen produced by a method with direct carbon emissions to the atmosphere; the first hydrogen gas portion and the second hydrogen gas portion are passed to the first reactor; and wherein, on average, a mass flow rate of the first hydrogen gas portion is at least 90% of a mass flow rate of the hydrogen gas product.
Aspect 20. The method of aspect 19, wherein the method to produce the first hydrogen gas portion comprises one or more of: utilization of renewable electricity; utilization of fossil fuels and sequestration of the produced carbon; or utilization of nuclear fission or fusion.

It should further be understood that streams may be named for the components of the stream, and the component for which the stream is named may be the major component of the stream (such as comprising from 50 weight percent (wt. %), from 70 wt. %, from 90 wt. %, from 95 wt. %, from 99 wt. %, from 99.5 wt. %, or even from 99.9 wt. % of the contents of the stream to 100 wt. % of the contents of the stream). It should also be understood that components of a stream are disclosed as passing from one system component to another when a stream comprising that component is disclosed as passing from that system component to another. For example, a disclosed “hydrotreated effluent stream” passing from a first system component to a second system component should be understood to equivalently disclose “propylene” passing from a first system component to a second system component, and the like.

For the purposes of describing and defining the present disclosure it is noted that the terms “about” or “approximately” are utilized in this disclosure to represent the inherent degree of uncertainty that may be attributed to any quantitative comparison, value, measurement, or other representation. The terms “about” and/or “approximately” are also utilized in this disclosure to represent the degree by which a quantitative representation may vary from a stated reference without resulting in a change in the basic function of the subject matter at issue.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

It is also noted that recitations herein of “at least one” component, element, etc., should not be used to create an inference that the alternative use of the articles “a” or “an” should be limited to a single component, element, etc.

What is claimed is:

1. A method of transporting hydrogen, the method comprising:
at a first chemical processing facility, passing carbon monoxide and hydrogen gas to a first reactor, wherein the carbon monoxide and the hydrogen gas undergo a Fischer-Tropsch reaction in the first reactor and form a mixed hydrocarbon product, wherein the mixed hydrocarbon product comprises linear alkanes;
transporting at least a portion of the mixed hydrocarbon product from the first chemical processing facility to a second chemical processing facility, wherein the first chemical processing facility and the second chemical processing facility are separated by a distance of at least 100 km; and
at the second chemical processing facility, dehydrogenating at least a portion of the mixed hydrocarbon product to form a hydrogen gas product.

2. The method of claim 1, wherein the mixed hydrocarbon product comprises at least 50 wt. % of alkanes, on the basis of the total weight of the mixed hydrocarbon product.

3. The method of claim 1, wherein at least 30 wt. % of the mixed hydrocarbon product is transported to the second chemical processing facility.

4. The method of claim 1, wherein at least 80 wt. % of the mixed hydrocarbon product is transported to the second chemical processing facility.

5. The method of claim 1, further comprising, at the first chemical processing facility, passing the mixed hydrocarbon product to a separation unit to form one or more hydrocarbon cuts, wherein transporting at least a portion of the mixed hydrocarbon product comprises transporting one or more of the hydrocarbon cuts.

6. The method of claim 5, wherein at least one of the hydrocarbon cuts that is transported to the second chemical processing facility is a first hydrocarbon cut that has an initial boiling point of from 20° C. to 100° C. and a final boiling point of from 180° C. to 220° C.

7. The method of claim 6, wherein the first hydrocarbon cut is dehydrogenated at the second chemical processing facility in a steam cracker to form the hydrogen gas product and light olefins.

8. The method of claim 1, wherein dehydrogenating the at least a portion of the mixed hydrocarbon product that is transported to the second chemical processing facility comprises steam cracking or catalytic reforming.

9. The method of claim 5, further comprising:
transporting at least one of the hydrocarbon cuts from the first chemical processing facility to a third chemical processing facility, wherein the first chemical processing facility and the third chemical processing facility are separated by a distance of at least 100 km; and
at the third chemical processing facility, dehydrogenating the one or more hydrocarbon cuts that are transported to the third chemical processing facility to form another hydrogen gas product.

10. The method of claim 9, wherein:
at least one of the hydrocarbon cuts that is transported to the third chemical processing facility is a second hydrocarbon cut; and
the second hydrocarbon cut comprises at least 50 wt. % of a combined weight of propane and butane, on the basis of the total weight of the second hydrocarbon cut.

11. The method of claim 1, wherein transporting the at least a portion of the mixed hydrocarbon product that is transported from the first chemical processing facility to the second chemical processing facility comprises moving the at least a portion of the mixed hydrocarbon product by truck, train, or ship.

12. The method of claim 1, wherein the at least a portion of the mixed hydrocarbon product that is transported from the first chemical processing facility to the second chemical processing facility is a liquid at a temperature of 20° C. and a pressure of 1 atmosphere.

13. The method of claim 1, wherein:
in the first reactor, the hydrogen and carbon monoxide are contacted with a Fischer-Tropsch catalyst comprising Fe, Co, or both; and
the first reactor is operated at a reaction temperature of from 150° C. to 300° C. and a reaction pressure of from 1 bar to 50 bar.

14. The method of claim 1, wherein the first reactor is a fixed-bed reactor or a fluidized bed reactor.

15. The method of claim 1, wherein:
the mixed hydrocarbon product exiting the first reactor comprises olefin compounds; and
the method further comprises, at the first chemical processing facility, hydrogenating at least a portion of the mixed hydrocarbon product that exits the first reactor upstream of transporting the least a portion of the mixed hydrocarbon product to the second chemical processing facility.

16. The method of claim 1, further comprising producing the carbon monoxide by partial oxidation of a hydrocarbon feed.

17. The method of claim 1, wherein the portion of the mixed hydrocarbon product that is transported from the first chemical processing facility to the second chemical processing facility does not undergo separation by boiling point at the first chemical processing facility.

18. The method of claim 1, further comprising, at the second chemical processing facility, passing the at least a portion of the mixed hydrocarbon product to a separator to form one or more hydrocarbon cuts, wherein one or more of the hydrocarbon cuts are dehydrogenated.

19. The method of claim 1, wherein:
a first hydrogen gas portion is hydrogen produced by a method with no direct carbon emissions to the atmosphere;
a second hydrogen gas portion is hydrogen produced by a method with direct carbon emissions to the atmosphere; the first hydrogen gas portion and the second hydrogen gas portion are passed to the first reactor; and
wherein, on average, a mass flow rate of the first hydrogen gas portion is at least 90% of a mass flow rate of the hydrogen gas product.

20. The method of claim 19, wherein the method to produce the first hydrogen gas portion comprises one or more of:
utilization of renewable electricity;
utilization of fossil fuels and sequestration of the produced carbon; or
utilization of nuclear fission or fusion.
